(19) United States
(12) Patent Application Publication
(10) Pub. No.: US 2025/0082515 A1
(43) Pub. Date: Mar. 13, 2025

(54) METHOD FOR PRODUCING A MULTILAYERED ABSORBENT PRODUCT AND PRODUCT THE METHOD

(71) Applicant: Avgol Industries 1953 LTD., Petah Tikva (IL)

(72) Inventors: Nick Carter, Cottonwood, AZ (US); DeeAnn Nelson, Mooresville, NC (US)

(21) Appl. No.: 18/576,996

(22) PCT Filed: Jul. 13, 2022
(86) PCT No.: PCT/IL2022/050751

§ 371 Date: Jan. 5, 2024

Related U.S. Application Data
(60) Provisional application No. 63/221,704, filed on Jul. 14, 2021.

(51) Int. Cl. A61F 13/15

(52) U.S. Cl. CPC A61F 13/15699

(57) ABSTRACT

The present invention is directed to a multilayered disposable absorbent article (24, 24A) and methods of manufacturing the same. In one form, the method includes the step of forming a base material or nonwoven substrate (12, 12A). The method further includes the step of applying a plurality of functional layers (14,14A; 16, 16A; 18, 18A; 20, 20A; 22, 22A) to the base material (12, 12A) to form a multilayered disposable absorbent article (24, 24A). Preferably, a release agent is included in one of the functional layers (14,14A; 16, 16A; 18, 18A; 20, 20A; 22, 22A) to which may be triggered chemically or mechanically to facilitate the removal of the functional layers (14,14A; 16, 16A; 18, 18A; 20, 20A; 22, 22A) from the base material during recycling of the article (24, 24A).

### CROSS-REFERENCE TO RELATED APPLICATION

The present application claims priority from U.S. Provisional Patent Application No. 63/221,704, filed on Jul. 14, 2021, the entire contents of which is incorporated herein by reference.

### TECHNICAL FIELD

The present invention relates generally to disposable absorbent articles, and more specifically to the manufacture of a disposable absorbent article having a multilayered, composite construction that is configurable based on a desired functional attributes of the article.

### BACKGROUND OF THE INVENTION

Nonwoven fabrics or substrates have been universally accepted and generally used in the construction of products exhibiting limited functional lifespans, such as components in disposable absorbent articles or products, and specifically as a performance barrier in baby diapers, feminine hygiene products and adult incontinence devices. Particularly preferred nonwoven barrier substrates include those produced by meltspun “direct formation” fabrics such as the combination of spunbond and meltblown technologies, wherein the nonwoven substrate is fabricated rapidly and at reduced complexity and cost as compared to nonwoven fabrics utilizing finite length staple fiber manufacturing means. Over time, and in the interest of minimizing both raw material consumption and waste production, industries utilizing direct formation nonwoven fabrics have requested that the nonwoven fabric exhibit nominal weight while achieving specified performance targets such as fluid absorption.

The successful production of a minimal mass, continuous filament nonwoven substrate is a compound problem comprising issues with formation of the continuous filament component to achieve required processing and end-use application physical and aesthetic performance attributes, suitable finite fluid management properties, and the attainment of these attributes by a viable commercial fabrication means wherein a nonwoven filamentous material exhibit useful aesthetic, physical and barrier qualities. Early prior art first address the means and methods of forming a basic spunmelt (as exemplified by spunbond and meltblown nonwoven technologies), such as is exemplified in U.S. Pat. Nos., 3,849,241 to Butin, et al., 3,855,046 to Hansen, 4,041,203 to Brock, et al. and 7,611,594 to Sommer et al. U.S. Patent No. 5,464,688 to Timmons et al., 5,662,978 and 6,100,208 to Brown et al. U.S. Patent No. 5,023,130 to Simpson et al., teaches a method by which unbonded continuous filaments are hydroentangled through application of high energy water jets. U.S. Pat. Nos. 7,858,544 and 8,093,163 to Turi, et al. offer an approach wherein to attain suitable filament movement and integration it is necessary to have either a low thermal point bond of less than 10% of the material surface area or an anisotropic bond pattern allowing for sufficient free filament length and engagement thereof. When one reviews the prior art more specific to end-use applications requiring high barrier performance, we again see the requirement for additional material layers which may be further formed into laminate or composite constructions. U.S. Pat. Nos. 5,888,614 to Slocum et al., 6,929,853 to Forte, 6,420,002 to Bonke, et al, 7,396,498 to Johnstone and both 6,602,809 and 6,706,225 to Cabrey each are directed to the use of films, either alone or in combination with one or more other substrates to achieve a high barrier performance material. U.S. Pat. No. 4,232,674 to Melican teaches the patterned deposition of an absorbent starch polymer in parallel stripes on the substrate of a sanitary towel. European Patent Application Publication No. 4,027,732 A1 to Hudson teaches ink jet printing onto absorbent articles. U.S. Pat. No. 9,108,787 of Schneider teaches various methods of printing graphics on absorbent products. Each of the aforementioned prior art patents are incorporated by reference in their respective entireties.

Nonwoven substrates may optionally include one or more other layers comprised of spunbond, conventional meltblown, films, stable fiber, cellulosic pulps, superabsorbent polymers (SAP) and the mixtures or combinations thereof for improved fluid management.

There remains an unmet need for the production of affordable disposable absorbent articles with improved recyclability and/or which exhibit improved control of the physical and aesthetic performance attributes, and fluid management properties, etc.

### SUMMARY OF THE INVENTION

The present invention is directed to a method of manufacturing a multilayered disposable absorbent article. The method includes the steps of forming a base material; and applying a plurality of functional layers to the base material to form a multilayered disposable absorbent article.

In a preferred form, the layers are applied to the base material by one of laser jet printing, gravure printing, digital printing, foam printing, additive manufacturing, and/or flexographic printing. Other printing or deposition means are contemplated.

According to another preferred form of the invention, the base material is one of a nonwoven substrate, a woven fabric, or a knitted fabric. Preferably, the multilayered disposable absorbent article is a diaper, a feminine hygiene product, or an adult incontinence device.

In one aspect of the present invention, the method further includes the step of including a release agent in at least one of the functional layers that is in direct contact with the base material. Preferably, the method further includes the step of applying a chemical or mechanical trigger to the multilayered disposable absorbent article to facilitate the separation of the functional layers from the base material during recycling or waste stream sorting. Specifically, separation of the base material from one or more of the functional layers, which may be composed of different materials having different levels of recyclability or different polymer recycling identification numbers (RINs), enhances sorting in the recycling stream. The chemical or mechanical trigger (i.e., release agent) may be applied by the user of the product containing the base material and the functional layers, or may be applied by the recycler.

In one broad form of the present invention, the functional layers include an absorbent layer, a liquid management layer, a skin contact layer, a breathable barrier layer, and a cushioning layer

In another broad form of the present invention, a multilayered disposable absorbent article is made according to the method or methods discussed above.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The invention will be more easily understood by a detailed explanation of the invention including drawings. Accordingly, drawings, which are particularly suited for explaining the inventions, are attached herewith; however, it should be understood that such drawings are for descriptive purposes only and as thus are not necessarily to scale beyond the measurements provided. The drawings are briefly described as follows:

FIG. 1 is a diagrammatic view of a first method or process of the present invention, and FIG. 1 shows a first exemplary nonwoven fabric according to the present invention undergoing treatment at a variety of stations to form a composite material exhibiting attributes of a conventional multiple component absorbent product; and

FIG. 2 is a diagrammatic view of a second method or process of the present invention, and FIG. 2 shows a second exemplary nonwoven fabric according to the present invention undergoing modification at a variety of stations, which layer/print one or more functional layers onto the base material containing a release agent which can be triggered to remove the functional layers from the base material for improved recycling.

### DETAILED DESCRIPTION OF THE INVENTION

While the present invention is susceptible of embodiment in various forms, there is shown in the drawings and will hereinafter be described a presently preferred embodiments of the invention, with the understanding that the present disclosure is to be considered as an exemplification of the invention, and is not intended to limit the broadest concepts of the invention to the specific embodiments illustrated. Apparatuses useful in preparing the filamentous material of the invention is conventional in nature and known to one skilled in the art. Such apparatuses include extruders, conveyor lines, water jets, rewinders or unwinders, topical applicators, calenders, compactors, and the like.

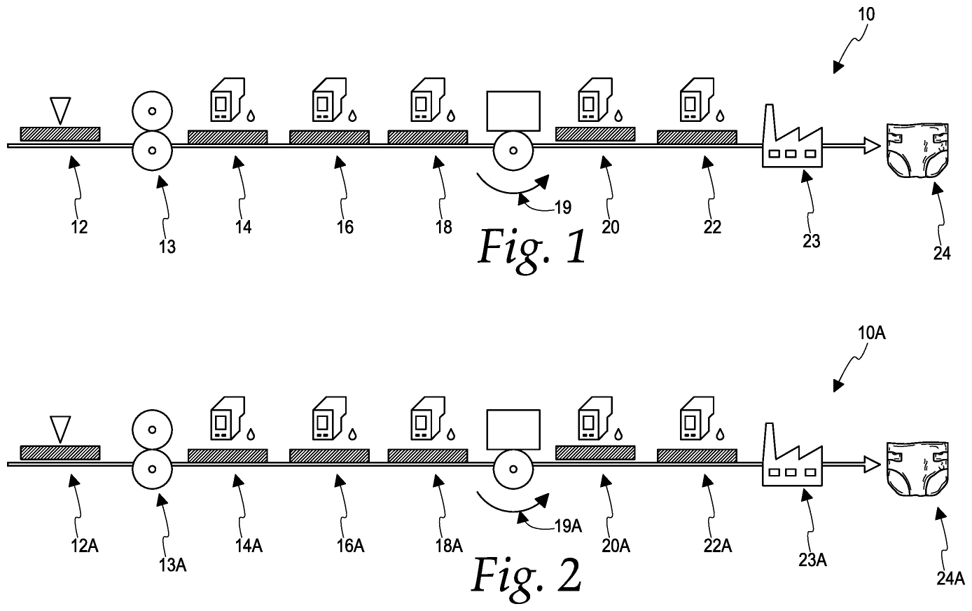

With reference to FIG. 1, a first embodiment of a method 10 of the present invention is illustrated diagrammatically. The method 10 includes the formation of a base material or substrate 12 in the form of a nonwoven fabric. Thermal calendaring 13 (indicated diagrammatically as a pair of opposing rollers) of the base material 12 is preferably performed, and then the base material 12 is then subjected to one or more of the multiple sequential or parallel stations which impart additional functional layers or attributes 14, 16, 18, 20, and 22 to the base material 12, such as an absorbent layer 14, a liquid management layer 16, a skin contact layer 18, a breathable barrier layer 20, a cushioning layer 22, etc. Other layers could be included. The base material 12 may be inverted 19 during processing to apply one or more of the layers 14, 16, 18, 20, and 22 to the other side or surface of the base material 12. The base material 12 with the layers 14, 16, 18, 20, and/or 22 may then be formed 23 into the resulting absorbent product or article 24 either indirectly (i.e., collected and shipped to the ultimate manufacturer of a consumer or end product) or directly formed in-line. The absorbent product or article 24 is a multilayered construct exhibiting the attributes of a conventional multiple component absorbent product of the prior art. The resulting absorbent product 24 may be a baby or toddler diaper, a feminine hygiene product, an adult incontinence device, etc. While FIG. 1 illustrates one exemplary or preferred embodiment of the present invention, it will be understood that the base material 12 (e.g., spunlaid nonwoven material) need not be made in line with the formation or deposition of the additional functional layers. The invention encompasses one or more steps of the method performed in parallel or series with respect to one or more other steps of the method, depending on the application. Other handling and/or finishing steps may be employed.

Base material 12 may be comprised of one or more of spunbond, meltblown, finite length fiber or integrated fiber constructs, pulp, tissues, woven scrims, etc. The functional layers 14, 16, 18, 20, 22 may be formed directly on the base material 12 by laser jet printing, gravure printing, digital printing, flexographic printing, foam printing, and/or additive manufacturing, etc. Other methods may be used.

In one form, the absorbent article 24 may include a permeable top sheet layer, or simply referred to as a “top sheet”, defining an interior surface of the article 24 in contact with the user of the article 24 and for receiving a liquid (e.g., urine, menstruation fluids, feces, or a mixture thereof). The top sheet may typically be formed from a nonwoven, such as a spunbond polymer, and can be comprised of any suitable relatively liquid-pervious material known in the art that permits passage of liquid therethrough such as non-woven spunbond or carded webs of polypropylene, polyethylene, nylon, polyester, natural cotton or bast fibers, and blends of these materials.

The absorbent article 24 will further typically include a liquid impermeable back sheet layer, or simply referred to as a “back sheet”, defining an exterior surface of the article 24 that faces away from the crotch of the user of the article 24. The back sheet may be formed from a polyethylene film or other liquid impermeable layers or films. For example, the back sheet may include materials such as a woven or non-woven material, a polymer film such as a polyethylene or polypropylene thermoplastic film, or a film-coated non-woven material.

The article 24 may optionally include any number of additional layers disposed between the top sheet and the back sheet, such as layers of highly lofted, wicking fibrous material layer that functions to quickly collect and temporarily hold discharged body fluids. Such layers may be formed from several different materials including nonwoven or woven webs of synthetic fibers including polyester, polypropylene, or polyethylene, natural fibers including cotton or cellulose, blends of such fibers, foams, fluff pulp, apertured films, or any equivalent materials or combinations of materials.

Importantly, the article 24 will include an absorbent core located between the top sheet and the back sheet. The core includes a substrate comprising filamentary components and more particularly to a filamentous material exhibiting useful fluid management barrier attributes while retaining aesthetic and physical performance necessary for mechanical processing of that material into useful and acceptable consumer products. The filamentous material includes at least one integrating network consisting essentially of continuous filaments formed from at least one polymeric material. Suitable polymeric materials include thermal melt and thermoset polymers, with thermal melt plastics being particularly preferred. The core may preferably be formed from a spunbond hydrophilic nonwoven manufactured by Avgol Ltd., having offices located at 9 Shimshon St., Lexus House, 7th floor, Petah Tikva, 4952707, Israel. Thermal melt plastics include polyolefins, and more preferably polypropylene or polyethylene. Other polymers suitable for use include polyesters, such as polyethylene terephthalate; polyamides; polyacrylates; polystyrenes; viscose, thermoplastic elastomers, block polymers, polymer alloys; and blends of these and other known fiber forming thermoplastic materials. The core may include one or more types of absorbent materials or superabsorbent polymers (“SAP”) distributed therein or thereupon. In one preferred form of the present invention, one or more of the base material and/or the SAP includes a superabsorbent polymer printed or otherwise deposited on the article 24. Preferably, the SAP is a biodegradable SAP. More preferably, the SAP is made from the polymers described in WO2019195271 A1 and/or WO2019195272 A1, which are incorporated by reference herein in their entireties. In one presently preferred form of the invention, the SAP is SAPgenix™ manufactured by Polygreen Group, having facilities at 3 Aluf Kalman Magen St., Tel Aviv 6107075, Israel.

With reference to FIG. 2, a second embodiment of a method 10A of the present invention is illustrated diagrammatically for producing an improved disposable absorbent article 24A, such as a baby or toddler diaper, a feminine hygiene product, an adult incontinence device, etc., having improved recyclability compared to state-of-the-art disposable absorbent products. The method 10A includes the formation of a base material or substrate 12A in the form of a nonwoven fabric. Thermal calendaring 13A (indicated diagrammatically as a pair of opposing rollers) of the base material 12A is preferably performed, and the base material 12A is then subjected to one or more of the multiple sequential or parallel stations which impart additional functional layers or attributes 14A, 16A, 18A, 20A, and 22A to the base material 12A, such as an absorbent layer 14A, a liquid management layer 16A, a skin contact layer 18A (which may be a function performed by the base material 12/12A or through separate application of a suitable skin contact chemistry, dissolvable fibrous layer or other types of fiber flocking), a breathable barrier layer 20A, a cushioning layer 22A, etc. Other layers could be included. The base material 12A may be inverted 19A during processing to apply one or more of the layers 14A, 16A, 18A, 20A, and 22A to the other side or surface of the base material 12A. The base material 12A with the layers 14A, 16A, 18A, 20A, and/or 22A may then be formed 23A into the resulting absorbent product or article 24A either indirectly (i.e., collected and shipped to the ultimate manufacturer of a consumer or end product) or directly formed in-line. The resulting absorbent product or article 24A is a multilayered construct exhibiting the attributes of a conventional multiple component absorbent product of the prior art and may be a baby or toddler diaper, a feminine hygiene product, an adult incontinence device, etc. While FIG. 2 illustrates another exemplary or preferred embodiment of the present invention, it will be understood that the base material 12A (e.g., spunlaid nonwoven material) need not be made in line with the formation or deposition of the additional functional layers. The invention encompasses one or more steps of the method performed in parallel or series with respect to one or more other steps of the method, depending on the application. Other handling and/or finishing steps may be employed.

The base material 12A may be comprised of one or more of spunbond, meltblown, finite length fiber or integrated fiber constructs, pulp, tissues, woven scrims, etc. The functional layers 14A, 16A, 18A, 20A, 22A may be formed directly on the base material 12A by laser jet printing, gravure printing, digital printing, foam printing, additive manufacturing, and/or flexographic printing, etc. Other methods may be used

Importantly, one or more of the layers 14A, 16A, 18A, 20A, and 22A that are in direct contact with the base material 12A further includes a release agent. The release agent, when triggered by an intentional trigger such as pH, chemical reagent, ultrasound, sunlight, etc., causes the functional layers 14A, 16A, 18A, 20A, and 22A on one or both surfaces of the base material 12A to become unengaged from the base material 12. By application of such chemical or chemical mechanical triggering conditions, the functional agents are removed, and the base material 12A may be recycled without contamination by the functional layers 14A, 16A, 18A, 20A, and 22A (which may not be the same material as the base material 12A).

From the foregoing, it will be observed that numerous modifications and variations can be effected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

1. A method of manufacturing a multilayered disposable absorbent article, said method comprising the steps of:
a. forming a base material; and
b. applying a plurality of functional layers to the base material to form a multilayered disposable absorbent article.

2. The method according to claim 1 wherein said step of applying a plurality of functional layers includes laser jet printing, gravure printing, digital printing, foam printing, additive manufacturing, and/or flexographic printing of said functional layers.

3. The method according to claim 1 further comprising the step of including a release agent in at least one of said functional layers that is in direct contact with said base material.

4. The method according to claim 3 further comprising the step of applying a chemical or mechanical trigger to said multilayered disposable absorbent article.

5. The method according to claim 4 further comprising the step of separating said plurality of functional layers from said base material.

6. The method according to claim 1 wherein said plurality of functional layers include an absorbent layer, a liquid management layer, a skin contact layer, a breathable barrier layer, and a cushioning layer.

7. The method according to claim 1 wherein said base material is one of a nonwoven substrate, a woven fabric, or a knitted fabric.

8. The method according to claim 1 wherein said disposable absorbent article is a diaper, a feminine hygiene product, or an adult incontinence device.

9. A disposable absorbent article made according to the method of claims 1,

10.-18. (canceled)
